(19) United States
(12) Patent Application Publication
(10) Pub. No.: US 2026/0272494 A1
(43) Pub. Date: Sep. 17, 2026

(54) DISSECTOR DEVICES WITH ILLUMINATION

(71) Applicant: Maduro Discovery, LLC, Los Gatos, CA (US)

(72) Inventors: Peter DAVIS, Dana Point, CA (US); Joshua Allen DAVISON, Buffalo, NY (US); Michael Thomas SZYMCZAK, Batavia, NY (US)

(73) Assignee: Maduro Discovery, LLC, Los Gatos, CA (US)

(21) Appl. No.: 19/672,305

(22) Filed: May 8, 2026

Related U.S. Application Data
(63) Continuation of application No. PCT/US2024/055109, filed on Nov. 8, 2024.
(60) Provisional application No. 63/597,253, filed on Nov. 8, 2023.

(51) Int. Cl. A61B 17/32; A61B 17/00; A61B 90/30; A61B 90/35

(52) U.S. Cl. CPC A61B 17/32; A61B 90/35; A61B 2017/00017; A61B 2017/00734; A61B 2017/320044; A61B 2090/309

(57) ABSTRACT

Dissectors for use in dissection during surgical procedures and having adjustable illumination profiles.

### CROSS-REFERENCE TO RELATED APPLICATIONS

This application is a continuation of International Patent Application Number PCT/US 2024/055109 filed Nov. 8, 2024, which claims priority to U.S. Provisional application 63/597,253 filed on Nov. 8, 2023, the entirety of which is incorporated by reference.

### BACKGROUND DESCRIPTION

Dissectors, including Penfield dissectors, are generally designed for use in dissection during surgical procedures and are especially useful in neurosurgical procedures. These dissectors can include a slightly flattened spatula-style head that allows the manipulating, lifting, and/or dissection of tissues.

### SUMMARY OF THE INVENTION

Variations of the present disclosure include a surgical dissector device having adjustable illumination profiles. For example, such dissector devices can include a handle body; a power supply; a shaft having a proximal portion coupled to the handle body, a medial portion away from the handle body and a dissector structure at a distal portion; where the dissector structure includes a dissector shape that is different than a remainder of the shaft and a blunt distal tip, where the dissector structure is configured to dissect tissue, the dissector shape including an opening; a first illumination source coupled to the power supply, the first illumination source is located within the opening, the first illumination source configured to provide a first illumination profile extending in a distal direction from the dissector structure; and a second illumination source located on the shaft, the second illumination source configured to provide a second illumination profile extending in the distal direction from the dissector structure, where the second illumination source can be adjusted to adjust a size or orientation of the second illumination profile.

In another variation, a surgical dissector device can include a bandpass filter located on the shaft, where the bandpass filter is configured to filter one or more wavelengths of light to accommodate visualization of tissue at or beyond the distal portion.

The dissector devices described herein can include the second illumination sources that are coupled to at least one shelf structure, where the at least one shelf structure is adjustable relative to the shaft to adjust the size or orientation of the second illumination profile. The shelf structure can be axially moveable along the shaft and/or deformable or deflectable relative to an axis of the shaft to adjust the size or orientation of the second illumination profile.

Variations dissector device can include an illumination source that emits a blue light that excites tissue loaded with 5-ALA. However, any wavelength of illumination is within the scope of this disclosure. In addition, the devices allow the illumination sources to change wavelength of illumination during the procedure.

The illumination sources can be an LED, any number of LEDs, and/or any source of illumination. Use of an LED configuration allows the device to emit various wavelengths of illumination.

The illumination sources can be coupled to its own switch on the device. For example, one or more switches can be located on the handle body, the shelf structure, and/or the shaft. One switch can control all the illumination sources. Alternatively, each illumination source can be coupled to an individual switch.

Variations of surgical dissector device include a power supply comprising includes at least one battery, either chargeable, or a DC battery. Variations can include the battery being in the handle body. Alternatively, or in combination, the power supply can be remote from the device/handle body.

The present disclosure also includes methods of identifying a region of tissue. For example, the method can include dissecting the region of tissue with a surgical dissector device including a shaft having a dissecting surface at a distal end and an illumination source coupled to the shaft, where the illumination source is configured to provide an illumination profile extending in a distal direction from the dissecting surface; and adjusting a size or direction of the illumination profile by adjusting the illumination source.

### DESCRIPTION OF THE DRAWINGS

Each of the following figures diagrammatically illustrates aspects of the invention. Variation of the invention from the aspects shown in the figures is contemplated.

FIG. 1 illustrates a variation of an improved surgical dissector.

FIG. 2A shows a front view of the device of FIG. 1 to illustrate placement of the illumination sources.

FIGS. 2B and 2C represent variations of illumination profiles.

FIGS. 3A and 3B provide illustrations of a view of devices taken from the handle looking towards the shelf 18 and distal end (not shown) through the bandpass filter.

### DETAILED DESCRIPTION

The present disclosure includes an improved Penfield dissection instrument that provides illumination of light of various wavelengths, either individually or simultaneously. The device can include one or more LEDs that emit a wavelength that effects the activation of a substance delivered to tissue. For example, the LED can emit blue light that excite tissue that has been previously loaded with 5-ALA, which is absorbed by tumors. The 5-ALA is a fluorescing drug such that the tumor can be exposed to an excitation wavelength of the light generated by the LED. Clearly, any spectrum of light can be used.

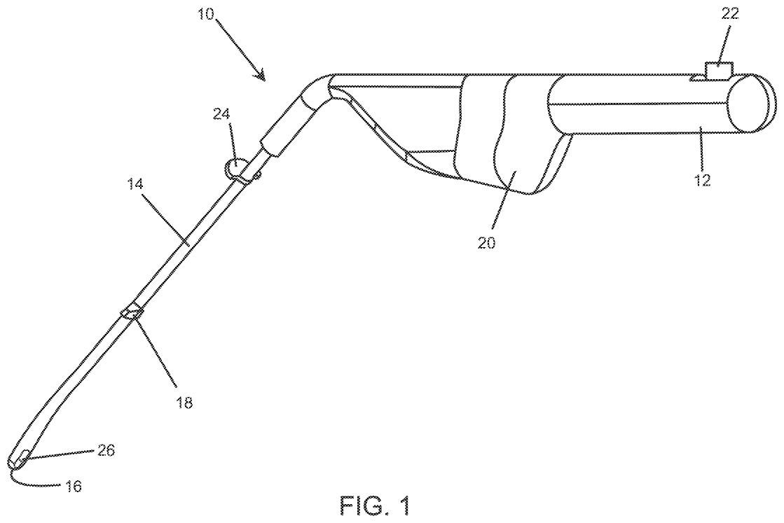

A variation of an improved Penfield instrument 10 is shown in FIG. 1. Generally, the device 10 can include a body or handle 12 with a shaft 14 extending away from the body 12. The distal end 16 of the shaft 14 can include a flattened portion, as is common with Penfield or other dissectors. Alternatively, the distal end 16 can include any shape required by the intended application for the device 10. The device includes one or more shelf features 18 that house illumination sources. For example, the illumination sources can comprise LEDs that emit illumination of various wavelengths. For example, the sources of illumination can include one or more switches 22 to power the illumination sources on or off as well as to switch between various wavelengths (e.g., 405 nm and white light). The shaft can include one or more lumens or passages to accommodate wiring as well as other structures described below. Variations of the device can include a compartment 20 for a power supply (e.g., batteries). Alternatively, the device 10 can rely on an external power supply.

FIG. 1 also shows a bandpass filter 24 on the shaft 14. The bandpass filter 24 is typically positioned proximally to the illumination sources to filter light from the illumination sources to accommodate visualization of tissue at the distal end 16 when using a camera or other imaging source viewing the distal end 16 from the handle end. In some variations of the device 10, the bandpass filter 24 is visually transparent or partially opaque to allow the operator to see through the filter 24. In one variation, the bandpass filter 24 is positioned distally to an illumination source if the illumination source is used for visible light or light of a wavelength that does not require filtering.

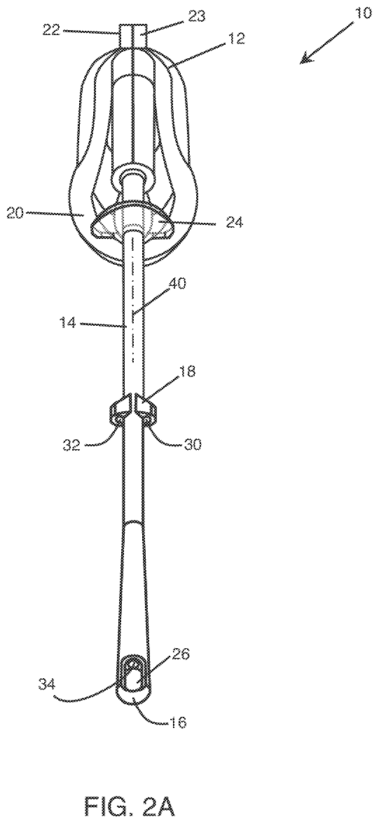

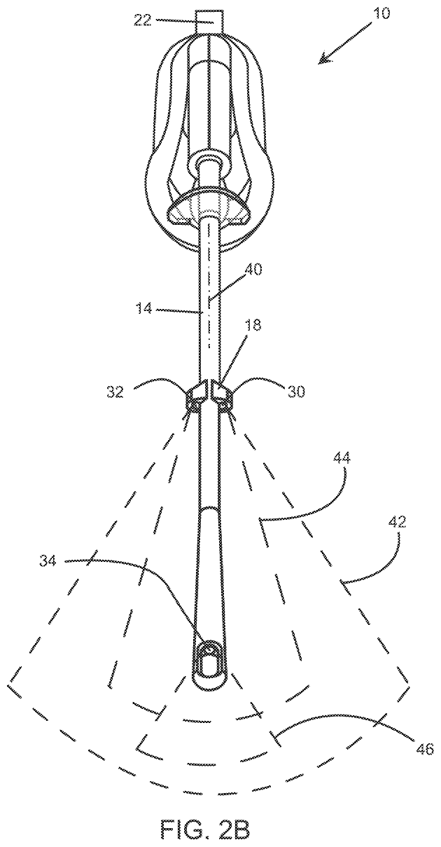

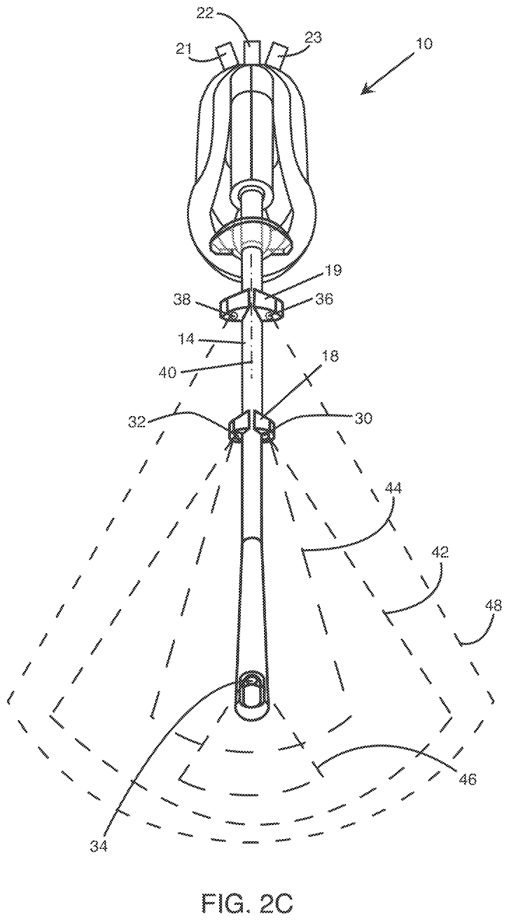

FIG. 2A shows a front view of the device 10 of FIG. 1 to better illustrate placement of the illumination sources (e.g., LEDs) 30, 32, 34. Any reference to LEDs in this disclosure is intended to include an alternate illumination source. As shown, two or more LEDs, 30, 32 can be positioned in the shelf 18 located on the shaft 14. Moreover, variations of the device 10 can include a number of shelves 18 positioned along the shaft 14 or body 12. Additionally, the illustrated variation shown in FIG. 2A includes an additional LED 34 located in the opening 26 at the distal end 16. Again, the device can include multiple LEDs at the opening.

One or more of the LEDS 30, 32, 34 can be configured to adjust an angle relative to an axis 40 of the shaft 14. This allows for increasing or decreasing the cone of illumination or can direct illumination to a specific area relative to the distal end 16 of the shaft 14. For example, LEDs 30, 32 can be adjustable manually (e.g., through deformation of the shelf 16) or through a deflecting mechanism as known in the art. Likewise, LED 34 can be adjusted manually or through a deflecting mechanism. In additional variations, the LEDS 30, 32, 34 can be moveable along the axis 40 to provide further customization of the illumination produced by each LED 30, 32, 34. Each LED can be adjusted individually or with one or more additional LEDs. It is also noted that any of the LEDs can be aligned at an angle to the axis 40 such that the illumination is focused distally, proximally, or laterally to the distal tip 16.

FIGS. 2B and 2C represent variations of illumination profiles 42, 44, 46. As shown, positioning of one or both of the LEDs 30, 32 in the shelf 18 can be changed to alter illumination profiles between 42 and 44. While LED 34 can produce illumination profile 46, which can also optionally be adjustable. As noted above, each LED can radiate the same wavelength or different wavelengths. It is noted that the LEDs can be controlled independently by one or more switches 22, 23 that control each illumination source or can control the wavelengths produced by the illumination sources. For example, each switch 22, 23 can control a separate illumination source and/or a wavelength of the illumination. Where toggling of the switch changes the wavelength. FIG. 2C shows multiple shelf structures 18, 19 spaced along the shaft 14 where each shelf structure 18, 19 includes one or more illumination sources 30, 32, 36, 38. It is noted that the same or different switches can also control the intensity of the illumination.

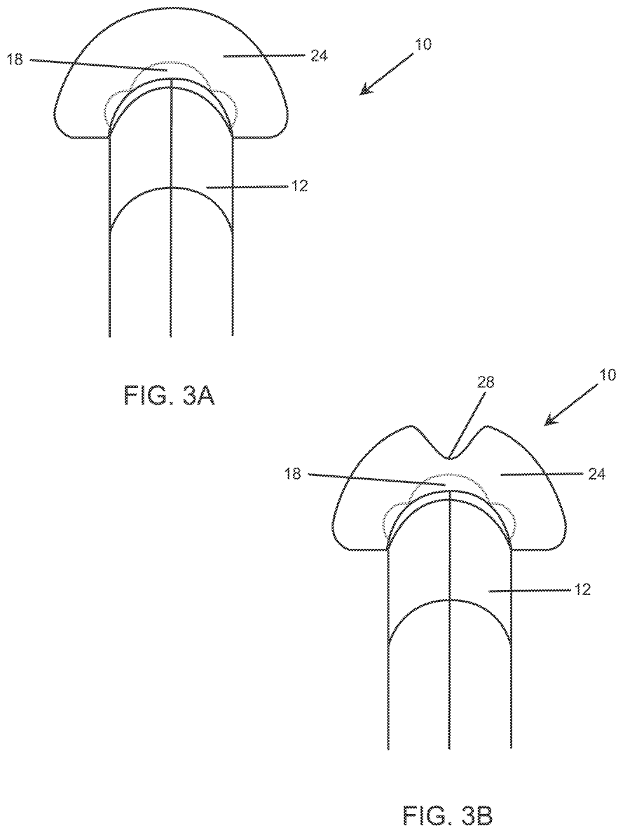

FIGS. 3A and 3B provide illustrations of a view of devices 10 taken from the handle 12 looking towards the shelf 18 and distal end (not shown) through the bandpass filter 24. As shown, the bandpass filter 24 can be transparent or partially opaque to allow viewing therethrough but to filter certain wavelengths of light as discussed above. In addition, FIG. 3B shows a bandpass filter 24 having a notch 28 to allow for positioning of one or more tools close to the shaft of the device 10 towards the distal end (not shown). Such a feature can be used for performing work removing, dissecting, or interrogating tissue or a tumor.

In one variation of a method of using the device, Indocyanine green (IcG) can be used to fluoresce blood vessels. The device could then be used to identify the blood vessels.

It is noted that any feature of a particular variation described above can be combined into alternate variations of the devices. As for other details of the present invention, materials and manufacturing techniques may be employed as within the level of those with skill in the relevant art. The same may hold true with respect to method-based aspects of the invention in terms of additional acts that are commonly or logically employed. In addition, though the invention has been described in reference to several examples, optionally incorporating various features, the invention is not to be limited to that which is described or indicated as contemplated with respect to each variation of the invention.

Various changes may be made to the invention described, and equivalents (whether recited herein or not included for the sake of some brevity) may be substituted without departing from the true spirit and scope of the invention. Also, any optional feature of the inventive variations may be set forth and claimed independently or in combination with any one or more of the features described herein. Accordingly, the invention contemplates combinations of various aspects of the embodiments or combinations of the embodiments themselves, where possible. Reference to a singular item, includes the possibility that there are plural of the same items present. More specifically, as used herein and in the appended claims, the singular forms “a,” “and,” “said,” and “the” include plural references unless the context clearly dictates otherwise.

It is important to note that where possible, aspects of the various described embodiments, or the embodiments themselves can be combined. Where such combinations are intended to be within the scope of this disclosure.

We claim:

1. A surgical dissector device, comprising:
a handle body;
a power supply;
a shaft having a proximal portion coupled to the handle body, a medial portion away from the handle body and a dissector structure at a distal portion;
where the dissector structure comprises a dissector shape that is different than a remainder of the shaft and a blunt distal tip, where the dissector structure is configured to dissect tissue, the dissector shape comprising an opening;
a first illumination source coupled to the power supply, the first illumination source is located within the opening, the first illumination source configured to provide a first illumination profile extending in a distal direction from the dissector structure; and
a second illumination source located on the shaft, the second illumination source configured to provide a second illumination profile extending in the distal direction from the dissector structure, where the second illumination source can be adjusted to adjust a size or orientation of the second illumination profile.

2. The surgical dissector device of claim 1, further comprising a bandpass filter located on the shaft, where the bandpass filter is configured to filter one or more wavelengths of light to accommodate visualization of tissue at or beyond the distal portion.

3. The surgical dissector device of claim 1, wherein the second illumination source is coupled to at least one shelf structure, where the at least one shelf structure is adjustable relative to the shaft to adjust the size or orientation of the second illumination profile.

4. The surgical dissector device of claim 3, where the at least one shelf structure is axially moveable along the shaft.

5. The surgical dissector device of claim 3, where the at least one shelf structure is deflectable relative to an axis of the shaft to adjust the size or orientation of the second illumination profile.

6. The surgical dissector device of claim 1, where at least the first illumination source or the second illumination source emits a blue light that excites tissue loaded with 5-ALA.

7. The surgical dissector device of claim 1, wherein the dissector shape comprises a flattened shape.

8. The surgical dissector device of claim 1, wherein the first illumination source and/or the second illumination source comprises an LED.

9. The surgical dissector device of claim 8, wherein the LED is configured to emit various wavelengths of illumination.

10. The surgical dissector device of claim 1, wherein the first illumination source is electrically coupled to a first switch on the handle body.

11. The surgical dissector device of claim 10, where the second illumination source is electrically coupled to the first switch.

12. The surgical dissector device of claim 10, where the second illumination source is electrically coupled to a second switch on the handle body.

13. The surgical dissector device of claim 1, wherein the power supply comprises at least one battery.

14. The surgical dissector device of claim 13, wherein the at least one battery is located in the handle body.

15. The surgical dissector device of claim 1, wherein the power supply is located remotely from the handle body.

16. A method of identifying a region of tissue, the method comprising:
dissecting the region of tissue with a surgical dissector device comprising a shaft having a dissecting surface at a distal end and an illumination source coupled to the shaft, where the illumination source is configured to provide an illumination profile extending in a distal direction from the dissecting surface; and
adjusting a size or direction of the illumination profile by adjusting the illumination source.
